(19) United States
(12) Patent Application Publication
(10) Pub. No.: US 2023/0050826 A1
(43) Pub. Date: Feb. 16, 2023

(54) METHOD AND APPARATUS FOR PRODUCING CANNABIS SMOKE RESIN

(71) Applicant: Thomas Alexander MEYER, Mississauga (CA)

(72) Inventor: Thomas Alexander MEYER, Mississauga (CA)

(21) Appl. No.: 17/401,594

(22) Filed: Aug. 13, 2021

(51) Int. Cl. A61K 36/185; A21D 13/06; A23L 33/105; A21C 15/00; A21D 13/24; A21D 13/28; A23B 4/052; A61K 9/00

(52) U.S. Cl. CPC A61K 36/185; A21D 13/06; A23L 33/105; A21C 15/00; A21D 13/24; A21D 13/28; A23B 4/052; A61K 9/0056; A23V 2002/00

(57) ABSTRACT

The present invention is a device and method for making Cannabis infused edibles comprising: a flow chamber having an inlet and an outlet, and configured to open; a porous material placed inside said flow chamber to form a tortuous flow path; a Cannabis burning chamber to burn Cannabis and generate a Cannabis smoke and a circulation system to circulate the Cannabis smoke in and out of the flow chamber. In operation one or more porous material are placed inside the flow chamber and infused with Cannabis smoke. The smoke passes continuously through the porous of said porous material and the resin of the smoke remains in the porous of the porous material; thereby produces a porous material which is smoke-resin infused for the purpose of consumption by means other than in-hailing smoke which is more convenient for consumers and has all the beneficial effects of Cannabis compounds.

### FIELD OF THE INVENTION

The present invention relates in general to Cannabis consumption and in specific to a method and process in which burnt Cannabis smoke is converted to resin for the purpose of consumption by means other than in-hailing smoke.

### BACKGROUND OF THE INVENTION

Cannabis is a psychoactive drug from the Cannabis plant used primarily for medical and recreational purposes. Smoking Cannabis is extremely harmful to the body as a means to consuming smoked Cannabis. It is harmful to the lungs, throat, mouth, etc. This harm is eliminated by offering a Cannabis smoke product that can be consumed another way rather than in-hailing smoke.

Cannabis consumption field consists of many forms which are in three main classes; smoking, vaporizing and eating. Both of vaporizing and eating Cannabis do not provide the same chemical ingredients as smoked Cannabis and as a consequence the intoxicating effect is drastically different. These forms of consumption do not provide the psychoactive intoxication which is characteristic of smoking Cannabis. Therefore, they fail to provide the consumer with the desired effect and they are not considered an adequate alternative. In order to get the desired effect one must consume the appropriate chemical ingredient which is smoked Cannabis.

Cannabis smoke harms the smoker's health. By in-hailing Cannabis smoke the body is exposed to an extremely harmful substance. Cannabis smoke consists of solid particulate matter (like dust) that includes a large number of organic and inorganic compounds. Some of these contents are harmful to the lungs, throat, mouth, etc. They can cause a varied number of conditions/diseases including cancer, specifically lung cancer.

Cannabis comprises various compounds. Among these compounds, tetrahydrocannabinol (THC), cannabinol (CBN), and cannabinodiol (CBDL) are known to be psychoactive. Other cannabinoids such as cannabidiol (CBD), which is a non-psychoactive compound, exert their physiological effects through a variety of receptors including adrenergic and cannabinoid receptors.

Many people may be reluctant to use Cannabis (either as a medicine or natural health product) if it has to be “smoked” or heated. This may be because there are negative perceptions to smoking and vaping, especially in the older generation. Many people may be more accepting of cannabis if it is in a form that is more conventional.

With the rise in the authorized use of cannabis for medical, therapeutic and recreational purposes, there is an increasing need for a reproducible method for obtaining a consistent active cannabinoid-comprising resin with known efficacy and to optimize production and recovery of active decarboxylated cannabinoids. With increasing demand and potential uses, there is also a need for a simple method for consumers.

In the recent years Cannabis is used in the smoking of foods including pure varieties of Cannabis, or hybrid varieties produced by crossing Cannabis sativa and Cannabis indicia. Cannabis is of interest for use in smoking foods because the plant contains cannabinoids, terpenoids, and other similar compounds that have known medical benefits for patients with a variety of ailments. Research has shown that Cannabis compounds can have beneficial health effects including reduction of pain, particularly neuropathic pain, treatment of chemotherapy-induced nausea and vomiting, and treatment of multiple sclerosis.

It is also being researched for its use in preventing seizures and reducing inflammation. An increasing number of medical trials are being conducted to research other beneficial effects of Cannabis compounds in a wide variety of ailments. Current research includes work to examine the effects of Cannabis on cancer, dementia, diabetes, epilepsy, glaucoma, Tourette's syndrome, ALS, and various digestive diseases.

The use of Cannabis to treat symptoms has typically been achieved by smoking the plant or vaporizing it. The inhalation of Cannabis smoke results in a tar being deposited into the lungs that is chemically similar to that of tobacco smoke, with over 50 known carcinogens present. Accordingly, it is desirable to identify other means of delivering the beneficial compounds of Cannabis to the consumers without the negative side-effects of smoking it.

This invention provides the consumer with a non-smoking method of consumption of Cannabis smoke. This is due to the consumer being able to consume the Cannabis smoke in another way in which the consumer benefits from the chemical composition needed and gets a much closer version of the desired effect.

### SUMMARY OF THE INVENTION

The present invention proposes a method and apparatus to produce a Cannabis product which is first smoked and then prepared for consumption in an edible form or any other non-smoking form. This invention discloses a process of converting burnt Cannabis smoke to resin for the purpose of consumption by means other than in-hailing smoke which is more convenient for consumers and has all the beneficial effects of Cannabis compounds.

Cannabis smoke is captured with the apparatus of the present invention which is a resin-collecting device (resinator). The device includes a flow chamber for containing a material for smoking, a smoke generator chamber is connected to the flow chamber for feeding and spouting the smoke into the flow chamber, and a vacuum pump connected to the flow chamber to circulate the Cannabis smoke in and out of the flow chamber.

The smoke generator spouts the smoke into the flow chamber to deposit smoke on the material inside the flow chamber. In one embodiment the material inside the flow chamber is a porous non-proteinaceous edible material which is compressively placed in the flow chamber. The smoke therefore permeates into the porous material and passes through the holes of the material. The process is repeated and the resin of the smoke remains in the porous of the material and can be consumed by the user.

The smoke generator is connected to the flow chamber by an inlet tube for feeding and spouting the smoke into the flow chamber. The vacuum means may comprise a vacuum pump or a fan connected to the flow chamber by an outlet tube for circulation of the Cannabis smoke.

Cannabis is smoked and immediately forced through the flow chamber which acts as a capturing device where all the smoke is held. The resin of the smoke remains in the porous of the material. With all the smoke ingredients captured by the material, it is now possible to prepare them appropriately for another method of consumption such as eating/drinking, chewing or skin absorbing.

Cannabis smoke resin is a brown colored and sticky material that tastes and smells disgusting and it is not particularly intuitive to any user of Cannabis that this resin is edible and beneficial. The Cannabis plant is capable of producing more than 400 different chemical compounds. Many of them are found in the smoke resin it produces. In fact, the primary psychoactive in the herb, THC, is contained in cannabis resin.

One of the main objects of this invention is to convert Cannabis smoke to Cannabis smoke resin as efficiently as possible and provide a process to make this product consumable. This method provides an “active” ingredient with the same chemical composition as smoked Cannabis. Smoked Cannabis has a different chemical composition of the active ingredient, THC, which has a distinct intoxicating effect. The intoxicating effect of smoked Cannabis is often referred to as a psychoactive effect which causes an increase in heart rate, and a pronounced psychoactive effect. All other methods of Cannabis consumption fail to produce the desired effect.

Another object of the present invention is to eliminate the need for in-hailing Cannabis smoke in order to achieve the desired effect. Due to the fact that the consumer no longer needs to in-hail the smoke, the consumer no longer experiences the harmful consequences of in-hailing the smoke.

Another object of the present invention is to provide medicinal Cannabis to the patients that can be consumed without smoking. This is significantly beneficial to the health of the patient.

Another object of the present invention is to provide a Cannabis product with the same benefit of the smoked Cannabis in every other non-smoking form for consumption (such as edibles).

It is yet another objective of the invention, to provide the Cannabis smoker with the “healthiest” option for the consumption of Cannabis smoke; which is the non-smoking consumption.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 is a perspective view of the device for making Cannabis infused edibles according to one embodiment of the present invention;

FIG. 2A is a cross-sectional view of the flow chamber according to FIG. 1;

FIG. 2B is a perspective view of the flow chamber according to FIG. 2A;

FIG. 3A is a cross-sectional view of the device for making Cannabis smoke resin according to another embodiment of the present invention;

FIG. 3B is a perspective view of the flow chamber according to FIG. 3A;

FIG. 4A is a cross-sectional view of the device for making Cannabis smoke resin according to another embodiment of the present invention, and

FIG. 4B is a perspective view of the flow chamber according to FIG. 4A.

### DETAILED DESCRIPTION OF PREFERRED EMBODIMENTS

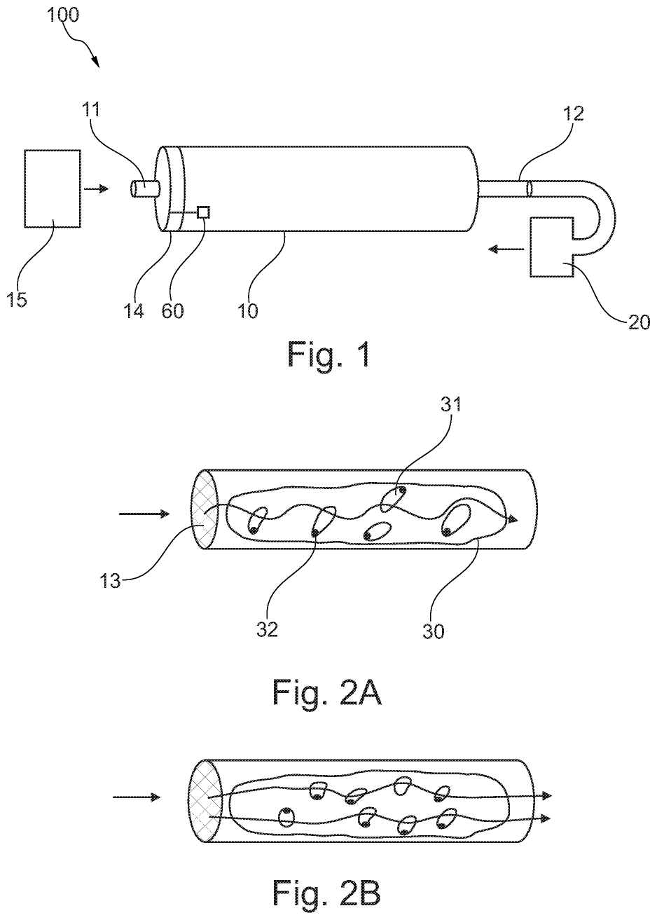

The present invention discloses a process of converting burnt Cannabis smoke to resin for the purpose of consumption by means other than in-hailing smoke. According to FIGS. 1 to 4B a device for making Cannabis infused edibles 100 in a simple version for this process is depicted. The device 100 consists of a cylindrical flow chamber 10 connected to an intake tube 11 for Cannabis smoke intake.

The flow chamber 10 leads to an air outlet tube 12. The flow chamber 10 is defined for containing a material to be infused with smoke resin which is produced by resination of the Cannabis smoke in the flow chamber 10. A smoke generator chamber 15 is connected to the intake tube 11 for feeding and spouting the smoke into the flow chamber 10. The intake tube 11 is designed to receive various smoke generator chambers that are used in Cannabis consumption. In one embodiment, a vacuum pump or a fan 20 is connected to the air outlet tube 12 to circulate the Cannabis smoke in and out of the flow chamber 10.

The smoke is generated from various forms of Cannabis smoking devices including but not limited to a pipe, vape, a rolled cigarette/joint, or any Cannabis burning device. Also, any type of Cannabis or Cannabis product (such as hash, oil, shatter, etc.) can be used to be burnt into smoke and fed into the intake tube 11.

The smoke is generated and fed into the flow chamber 10. The flow of air is maintained in the direction of intake tube 11 to the chamber 10 and from the chamber 10 to the outlet tube 12. The vacuum/fan 20 is affixed to the air outlet tube 12 which draws the air to the outlet tube 12 from the intake tube 11. The Cannabis smoke is fed to the intake tube 11 with a constant direction of air going from intake tube 11 to outlet tube 12 and the smoke travels through the flow chamber 10 in between. The flow system forces the Cannabis smoke through the porous material inside the flow chamber, whereby the Cannabis smoke resin infuses and coats the the porous material and the Cannabis smoke resin is collected in the pores of the porous material.

In one embodiment as depicted in FIGS. 1, 2A and 2B the device 100 is intended to process a porous non-proteinaceous edible material like a loaf of bread 30. Bread 30 is placed inside the flow chamber 10 to be infused with Cannabis smoke. Various possible options can be designed to load the flow chamber 10. In one embodiment the chamber 10 comprises of a lid 14 on top portion for placing the material to be infused inside the flow chamber 10.

Edible materials similar to bread 30 provide porous and holes which build a passage for smoke to travel through each passage, therefore, providing many passages all collecting smoke resin in between the porous. The smoke is forced towards the bread 30 and flow through the bread and out of the output tube 12. The smoke travels through each passage and the smoke resin 32 is collected in each porous 31 of the edible. This process results that Cannabis smoke exhibits resin-collecting or resination. A set of screen 13 is provided on the distal end and the proximal end of the flow chamber 10 to prevent the edible 30 to be forced out of the chamber 10 into the outlet tube 12.

When Cannabis smoke is incident on a solid surface it begins to collect resin on that surface. By forcing Cannabis smoke through a tight hole or crack, it will resinate on the surfaces of the crack to produce a collecting resin pool which eventually clogs the hole or crack. So the Cannabis smoke travels through the flow chamber 10 collecting in many cracks and holes along the way. If there are enough cracks and holes, all of the Cannabis smoke will resinate in the chamber and the air that exits from the output will be free of smoke. This is the ideal case, that there is enough collection of resin 32 occurring in the flow chamber 10 to ensure that all of the smoke is efficiently collected as resin 32.

The longer and wider the chamber 10 is, the better it functions. However, if the chamber is too long, then it becomes more difficult to draw the air. Therefore, the proper dimension in design of the device 100 should be considered. At the end of the process the combined contents of bread and resin are the final product. Unless the user wishes to separate the resin 32 from the edible 30, it is assumed that the combined product is edible. This implies that in most cases the resulting product is to be consumed by eating.

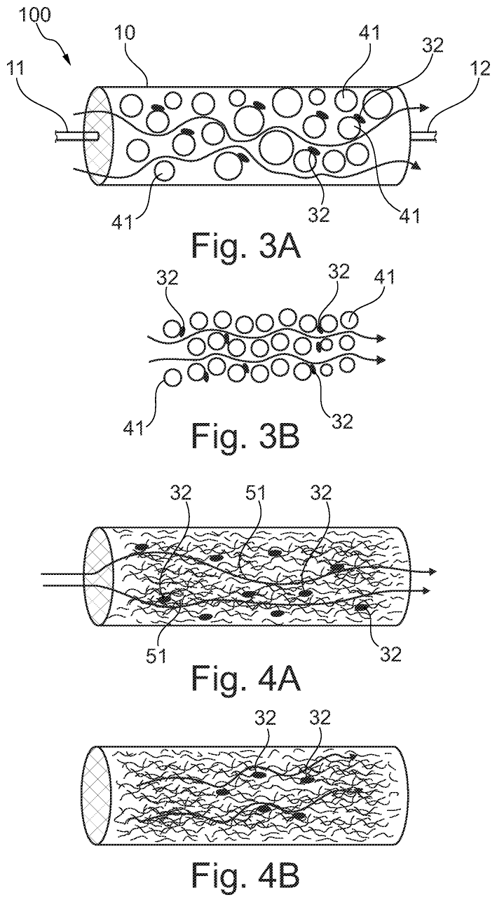

In another embodiment which is depicted in FIGS. 3A to 4B the flow chamber 10 is filled with a solid object to sufficiently minimize the flow of air and build air path ways so that the smoke can pass through the air passages and collect resin.

In one embodiment the solid object comprises of a plurality of packed beads 41 forming a porous passage. The flow chamber 10 is filled with beads 41 to create many small passages of air similar to porous. The beads 41 maximally fill the flow chamber 10 to build many routes for travel of air and deposit smoke resin 32 on the surfaces of the plurality of packed beads 41. The hole dimensions are sufficiently small for resin collection. The amount of the beads 41 to place inside the flow chamber 10 has to be selected properly. Then, the combined beads and smoke resin can be removed from the chamber 10 and the application of heat will separate the beads 41 from the viscous resin 32 for edible purposes.

According to FIGS. 4A and 4B in another embodiment the solid object is selected from steel wool 51 which is flexible and easy to fit into the flow chamber 10. Steel wool 51 has many passages of air travel there-through which can be optimized by choosing how much wool is used for a given size of flow chamber 10. The smoke resin 32 is deposited on the porous of the steel wool 51 and can be removed from the steel wool for edible purposes. The combined mixture of resin 32 and steel wool 51 can be removed from the flow chamber 10 and application of heat to the mixture will separate the viscous resin 32 from the steel wool 51 for edible purposes.

The dimension for passage holes is roughly 1 mm, so that smoke can travel through each passage and that resin will collect in each passage, so with many passages all collecting resin 32 to achieve an efficient collection of resin. If the average diameter of each passage hole is too large then there will be less resin collection in that passage, meaning there will be less efficient resin collection. Therefore, the amount of the steel wool 51 to place inside the flow chamber 10 has to be selected properly.

The operating temperature is to be optimized for the device operation. If the temperature chosen for operation is between 0° C. and room temperature, then the device will operate properly. If the temperature is much too high, above 170° C., then the active ingredient (THC) vaporizes and escapes. Therefore, the ideal operating temperature is chosen so that the viscous resin will efficiently collect on the surface of the material.

According to FIG. 1 again the flow chamber 10 may include a temperature sensor 60 for detecting the temperature in the chamber and to adjust and maintain the temperature within a predetermined range of temperature.

The device is made from various materials including but not limited to metal which is non-melting and non-radiating at the operational temperatures. In a preferred embodiment all of the component parts including the intake and outlet tubes 11 and 12, flow chamber 10, screen 13 and solid object 41 and 51 are made of stainless steel, however, other suitable materials appropriate for the operational temperature range can be used in construction of the device 100.

The collected Cannabis smoke resin 32 can be extracted from the porous solid material for consumption in various methods. In one method, extracting the Cannabis smoke resin is through removing the porous solid material from the flow chamber and heating it to extract the Cannabis smoke resin. In another method, extracting the Cannabis smoke resin is through heating the porous solid material while inside the flow chamber and extracting a liquid resin from the bottom part of the flow chamber.

The Cannabis smoke resin 32 can be heated up to a temperature to get a liquid product. The non-active ingredients can be filtered from the resin by various filters such as a carbon filter.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1) A method for making a Cannabis infused edible, comprising the steps of:
a) placing a porous non-proteinaceous edible material inside a flow chamber;
b) making a Cannabis smoke containing Cannabis smoke resin by burning Cannabis in a Cannabis smoke generator chamber;
c) passing the Cannabis smoke through the flow chamber to infuse said porous non-proteinaceous edible material with the Cannabis smoke resin to make the Cannabis infused edible.

2) A method for making a Cannabis infused edible, comprising the steps of:
d) placing a porous solid material inside a flow chamber;
e) making a Cannabis smoke containing Cannabis smoke resin by burning Cannabis in a Cannabis smoke generator chamber;
f) passing the Cannabis smoke through the flow chamber to coat the pores of said solid material with the Cannabis smoke resin;
g) extracting the Cannabis smoke resin from the porous solid material for consumption.

3) The method of claim 2, wherein extracting the Cannabis smoke resin is through removing the porous solid material from the flow chamber and heating it to extract the Cannabis smoke resin.

4) The method of claim 2, wherein extracting the Cannabis smoke resin is through heating the porous solid material while inside the flow chamber and extracting a liquid resin from the bottom part of the flow chamber.

5) An apparatus for making Cannabis infused edibles comprising:
a) a flow chamber having an inlet, an outlet, and configured to receive a porous material;
b) a Cannabis burning chamber connected to the inlet of said flow chamber to burn Cannabis and generate a Cannabis smoke containing a Cannabis smoke resin, and
c) a flow system to force the Cannabis smoke through the porous material inside the flow chamber, whereby the Cannabis smoke resin infuses and coats the the porous material and the Cannabis smoke resin is collected in the pores of the porous material.

6) The apparatus for making Cannabis infused edibles of claim 5, wherein the porous material is a porous non-proteinaceous edible.

7) The apparatus for making Cannabis infused edibles of claim 5, wherein the porous material is a steel wool.

8) The apparatus for making Cannabis infused edibles of claim 5, wherein the porous material is made of a plurality of packed beads forming a porous passage, whereby the Cannabis smoke resin deposits on the surfaces of said plurality of packed beads, and the Cannabis smoke resin is extracted by heating the plurality of packed beads.

9) The apparatus for making Cannabis infused edibles of claim 5, wherein the flow system comprises of a fan, or a vacuum pump attached to the outlet of the flow chamber.

10) The apparatus for making Cannabis infused edibles of claim 5, wherein the flow chamber further having a temperature sensor for detecting the temperature inside said flow chamber and to adjust and maintain the temperature within a predetermined range of temperature.
